(19) United States
(12) Patent Application Publication
(10) Pub. No.: US 2024/0120685 A1
(43) Pub. Date: Apr. 11, 2024

(54) ELECTRICAL CONNECTOR WITH TORSION RESISTANCE

(71) Applicant: Cochlear Limited, New South Wales (AU)

(72) Inventor: Eddie Sze Chuen Chan, Macquarie University (AU)

(21) Appl. No.: 18/257,713

(22) PCT Filed: Jan. 3, 2022
(86) PCT No.: PCT/IB2022/050027

§ 371 Date: Jun. 15, 2023

Related U.S. Application Data
(60) Provisional application No. 63/136,368, filed on Jan. 12, 2021.

(51) Int. Cl. H01R 13/629; A61N 1/36; A61N 1/375; H01R 13/11

(52) U.S. Cl. CPC H01R 13/629; A61N 1/36038; A61N 1/3752; H01R 13/112; H01R 13/03; H01R 2201/12

(57) ABSTRACT

An apparatus includes a first portion and a second portion configured to be repeatedly mechanically coupled to and decoupled from the first portion. The first portion includes at least three protrusions in electrical communication with first circuitry. Each of the at least three protrusions is displaced from and extends substantially parallel to a center axis and at least two of the at least three protrusions are displaced from the center axis by distances that are substantially different from one another. The second portion includes at least three receptacles in electrical communication with second circuitry. Each of the at least three receptacles includes two tines configured to be in mechanical and electrical communication with a corresponding protrusion of the at least three protrusions upon insertion of the corresponding protrusion into a region at least partially bounded by the two tines. The two tines are displaced from and extend substantially parallel to the center axis and are configured to inhibit a relative rotation between the first portion and the second portion about the center axis.

### BACKGROUND

### Field

The present application relates generally to systems and methods for facilitating wired power and data transmission, and more specifically, for facilitating wired power and data transmission using two connector portions configured to be repeatedly mechanically coupled to and decoupled from one another.

### Description of the Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect disclosed herein, an apparatus comprises a first portion and a second portion configured to be repeatedly mechanically coupled to and decoupled from the first portion. The first portion comprises at least three protrusions in electrical communication with first circuitry. Each of the at least three protrusions is displaced from and extends substantially parallel to a center axis, at least two of the at least three protrusions displaced from the center axis by distances that are substantially different from one another. The second portion comprises at least three receptacles in electrical communication with second circuitry. Each of the at least three receptacles comprises two tines configured to be in mechanical and electrical communication with a corresponding protrusion of the at least three protrusions upon insertion of the corresponding protrusion into a region at least partially bounded by the two tines. The two tines are displaced from and extend substantially parallel to the center axis and are configured to inhibit a relative rotation between the first portion and the second portion about the center axis.

In another aspect disclosed herein, an apparatus comprises at least three electrically conductive forks spaced from and distributed about an axis. Each fork of the at least three forks has a pair of substantially parallel prongs that extend substantially parallel to the axis and are spaced from one another along a line substantially perpendicular to the axis and substantially perpendicular to a direction extending from the axis to the fork. In certain such aspects, the apparatus further comprises at least three electrically conductive pins spaced from and distributed about the axis and extending substantially parallel to the axis. The at least three pins are configured to repeatedly mechanically and electrically engage with and disengage from the at least three forks. At least two of the at least three electrically conductive forks are displaced from the axis by distances that are substantially different from one another.

In another aspect disclosed herein, a method comprises providing a first mating portion comprising a plurality of electrically conductive pins and a second mating portion comprising a plurality of electrically conductive forks configured to receive the plurality of electrically conductive pins. The method further comprises mating the first mating portion with the second mating portion such that each of the forks of the plurality of forks is in electrical and mechanical communication with a corresponding pin of the plurality of pins. The method further comprises, in response to an applied torque between the first and second mating portions, using each fork to block movement of the corresponding pin.

### BRIEF DESCRIPTION OF THE DRAWINGS

Implementations are described herein in conjunction with the accompanying drawings, in which:

FIG. 1 is a perspective view of an example cochlear implant auditory prosthesis implanted in a recipient in accordance with certain implementations described herein;

FIG. 2 schematically illustrates an example apparatus in accordance with certain implementations described herein;

FIGS. 3A-3D schematically illustrate top views of example apparatus along the center axis in accordance with certain implementations described herein.

FIG. 4 schematically illustrates an example system comprising an example apparatus in accordance with certain implementations described herein;

FIGS. 5A-5B schematically illustrate substantially cross-sectional views of an example apparatus with the first portion and the second portion mechanically decoupled from one another and mechanically coupled to one another, respectively, in accordance with certain implementations described herein;

FIG. 6A schematically illustrates a perspective view of an example second portion in accordance with certain implementations described herein;

FIG. 6B schematically illustrates three side views of the example second portion of FIG. 6A;

FIG. 6C schematically illustrates a substantially cross-sectional view in a plane extending along an example protrusion and an example receptacle in mechanical and electrical communication with one another in accordance with certain implementations described herein;

FIG. 7 schematically illustrates a substantially cross-sectional view in a plane substantially perpendicular to a center axis extending through an example apparatus in accordance with certain implementations described herein;

FIGS. 8A-8B schematically illustrate two example second portions having at least one rotation inhibiting structure in accordance with certain implementations described herein; and

FIG. 9 is a flow diagram of an example method in accordance with certain implementations described herein.

### DETAILED DESCRIPTION

Certain implementations described herein provide small electrical multi-pin connectors (e.g., for use in wearable devices or medical devices) that provide enhanced torsion resistance (e.g., up to 25 N-cm) without compromising component size, electrical conductivity, and/or sealing. A first portion of the connector comprises a plurality of pins and a second portion of the connector comprises a plurality of electrically conductive forks having prongs configured to generate a counter-torque to an externally-applied relative torque between the first and second portions. For example, the electrical connector of certain implementations can provide improved torsion resistance (e.g., inhibiting damage such as bending of the pins and/or forks under the externally-applied relative torque), while keeping the size of the connector relatively small.

The teachings detailed herein are applicable, in at least some implementations, to any type of system or device (e.g., medical device configured to be worn by a recipient) having two electrical connector portions expected to be repeatedly mechanically coupled to and decoupled from one another and to undergo and withstand torques applied between the two portions. For example, the system can be an implantable medical device (e.g., implantable sensory prostheses; auditory prosthesis system) comprising an external first sub-system (e.g., sound processor external to a recipient) and an internal second sub-system (e.g., actuator and/or stimulator implanted on or within the recipient and configured to generate stimulation signals that are perceived by the recipient as sounds). The first sub-system can comprise two electrical connector portions (e.g., a first electrical connector portion that is a component of the external sound processor and a second electrical connector portion that is a component of an electrical cable in operative communication with an external communication unit (e.g., communication coil) configured to wirelessly (e.g., transcutaneously) transmit power and/or data (e.g., control signals) to the second sub-system and to wirelessly (e.g., transcutaneously) communicate with the second sub-system. Examples of auditory prosthesis systems compatible with certain implementations described herein include but are not limited to: electro-acoustic electrical/acoustic systems, cochlear implant devices, implantable hearing aid devices, middle ear implant devices, Direct Acoustic Cochlear Implant (DACI), middle ear transducer (MET), electro-acoustic implant devices, other types of auditory prosthesis devices, and/or combinations or variations thereof, or any other suitable hearing prosthesis system with or without one or more external components Implementations can include any type of medical device that can utilize the teachings detailed herein and/or variations thereof.

Merely for ease of description, apparatus and methods disclosed herein are primarily described with reference to an illustrative medical device, namely a cochlear implant. However, the teachings detailed herein and/or variations thereof may also be used with a variety of other wearable components/devices (e.g., medical devices) that provide a wide range of therapeutic benefits to recipients, patients, or other users. In some implementations, the teachings detailed herein and/or variations thereof can be utilized in other types of implantable medical devices beyond auditory prostheses. For example, apparatus and methods disclosed herein and/or variations thereof may also be used with one or more of the following: vestibular devices (e.g., vestibular implants); visual devices (e.g., bionic eyes); visual prostheses (e.g., retinal implants); sensors; cardiac pacemakers; drug delivery systems; defibrillators; functional electrical stimulation devices; catheters; brain implants; seizure devices (e.g., devices for monitoring and/or treating epileptic events); sleep apnea devices; electroporation; etc. The concepts described herein and/or variations thereof can be applied to any of a variety of implantable medical devices comprising an implanted component configured to use magnetic induction to communicate transcutaneously with an external component (e.g., receive control signals from the external component and/or transmit sensor signals to the external component) while using magnetic induction to receive power from the external component. In still other implementations, the teachings detailed herein and/or variations thereof can be utilized in other types of systems beyond components/devices (e.g., medical devices) utilizing magnetic induction for both wireless power transfer and data communication. For example, such other components, devices, and/or systems can include one or more of the following: wearable devices (e.g., smartwatches), consumer products (e.g., smartphones; IoT devices), and electric vehicles (e.g., automobiles).

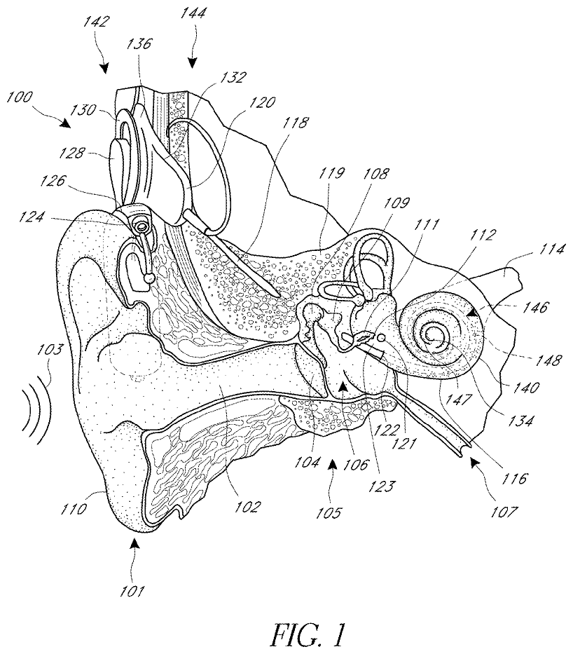

FIG. 1 is a perspective view of an example cochlear implant auditory prosthesis 100 implanted in a recipient in accordance with certain implementations described herein. The example auditory prosthesis 100 is shown in FIG. 1 as comprising an implanted stimulator unit 120 (e.g., an actuator) and an external microphone assembly 124 (e.g., a partially implantable cochlear implant). An example auditory prosthesis 100 (e.g., a totally implantable cochlear implant) in accordance with certain implementations described herein can replace the external microphone assembly 124 shown in FIG. 1 with a subcutaneously implantable assembly comprising an acoustic transducer (e.g., microphone), as described more fully herein.

As shown in FIG. 1, the recipient normally has an outer ear 101, a middle ear 105, and an inner ear 107. In a fully functional ear, the outer ear 101 comprises an auricle 110 and an ear canal 102. An acoustic pressure or sound wave 103 is collected by the auricle 110 and is channeled into and through the ear canal 102. Disposed across the distal end of the ear canal 102 is a tympanic membrane 104 which vibrates in response to the sound wave 103. This vibration is coupled to oval window or fenestra ovalis 112 through three bones of middle ear 105, collectively referred to as the ossicles 106 and comprising the malleus 108, the incus 109, and the stapes 111. The bones 108, 109, and 111 of the middle ear 105 serve to filter and amplify the sound wave 103, causing the oval window 112 to articulate, or vibrate in response to vibration of the tympanic membrane 104. This vibration sets up waves of fluid motion of the perilymph within the cochlea 140. Such fluid motion, in turn, activates tiny hair cells (not shown) inside the cochlea 140. Activation of the hair cells causes appropriate nerve impulses to be generated and transferred through the spiral ganglion cells (not shown) and auditory nerve 114 to the brain (also not shown) where they are perceived as sound.

As shown in FIG. 1, the example auditory prosthesis 100 comprises one or more components which are temporarily or permanently implanted in the recipient. The example auditory prosthesis 100 is shown in FIG. 1 with an external component 142 which is directly or indirectly attached to the recipient's body, and an internal component 144 which is temporarily or permanently implanted in the recipient (e.g., positioned in a recess of the temporal bone adjacent auricle 110 of the recipient). The external component 142 typically comprises one or more input elements/devices for receiving input signals at a sound processing unit 126. The one or more input elements/devices can include one or more sound input elements (e.g., one or more external microphones 124) for detecting sound and/or one or more auxiliary input devices (not shown in FIG. 1)(e.g., audio ports, such as a Direct Audio Input (DAI); data ports, such as a Universal Serial Bus (USB) port; cable ports, etc.). In the example of FIG. 1, the sound processing unit 126 is a behind-the-ear (BTE) sound processing unit configured to be attached to, and worn adjacent to, the recipient's ear. However, in certain other implementations, the sound processing unit 126 has other arrangements, such as by an OTE processing unit (e.g., a component having a generally cylindrical shape and which is configured to be magnetically coupled to the recipient's head), a mini or micro-BTE unit, an in-the-canal unit that is configured to be located in the recipient's ear canal, a body-worn sound processing unit, etc.

The sound processing unit 126 of certain implementations includes a power source (not shown in FIG. 1)(e.g., battery), a processing module (not shown in FIG. 1)(e.g., comprising one or more digital signal processors (DSPs), one or more microcontroller cores, one or more application-specific integrated circuits (ASICs), firmware, software, etc. arranged to perform signal processing operations), and an external transmitter unit 128. In the illustrative implementation of FIG. 1, the external transmitter unit 128 comprises circuitry that includes at least one external inductive communication coil 130 (e.g., a wire antenna coil comprising multiple turns of electrically insulated single-strand or multi-strand platinum or gold wire). The external transmitter unit 128 also generally comprises a magnet (not shown in FIG. 1) secured directly or indirectly to the at least one external inductive communication coil 130. The at least one external inductive communication coil 130 of the external transmitter unit 128 is part of an inductive radio frequency (RF) communication link with the internal component 144. The sound processing unit 126 processes the signals from the input elements/devices (e.g., microphone 124 that is positioned externally to the recipient's body, in the depicted implementation of FIG. 1, by the recipient's auricle 110). The sound processing unit 126 generates encoded signals, sometimes referred to herein as encoded data signals, which are provided to the external transmitter unit 128 (e.g., via a cable). As will be appreciated, the sound processing unit 126 can utilize digital processing techniques to provide frequency shaping, amplification, compression, and other signal conditioning, including conditioning based on recipient-specific fitting parameters.

The power source of the external component 142 is configured to provide power to the auditory prosthesis 100, where the auditory prosthesis 100 includes a battery (e.g., located in the internal component 144, or disposed in a separate implanted location) that is recharged by the power provided from the external component 142 (e.g., via a transcutaneous energy transfer link). The transcutaneous energy transfer link is used to transfer power and/or data to the internal component 144 of the auditory prosthesis 100. Various types of energy transfer, such as infrared (IR), electromagnetic, capacitive, and inductive transfer, may be used to transfer the power and/or data from the external component 142 to the internal component 144. During operation of the auditory prosthesis 100, the power stored by the rechargeable battery is distributed to the various other implanted components as needed.

The internal component 144 comprises an internal receiver unit 132, a stimulator unit 120, and an elongate stimulation assembly 118. In some implementations, the internal receiver unit 132 and the stimulator unit 120 are hermetically sealed within a biocompatible housing, sometimes collectively referred to as a stimulator/receiver unit. The internal receiver unit 132 comprises at least one internal inductive communication coil 136 (e.g., a wire antenna coil comprising multiple turns of electrically insulated single-strand or multi-strand platinum or gold wire), and generally, a magnet (not shown in FIG. 1) fixed relative to the at least one internal inductive communication coil 136. The at least one internal inductive communication coil 136 receives power and/or data signals from the at least one external inductive communication coil 130 via a transcutaneous energy transfer link (e.g., an inductive RF link). The stimulator unit 120 generates stimulation signals (e.g., electrical stimulation signals; optical stimulation signals) based on the data signals, and the stimulation signals are delivered to the recipient via the elongate stimulation assembly 118.

The elongate stimulation assembly 118 has a proximal end connected to the stimulator unit 120, and a distal end implanted in the cochlea 140. The stimulation assembly 118 extends from the stimulator unit 120 to the cochlea 140 through the mastoid bone 119. In some embodiments, the stimulation assembly 118 can be implanted at least in the basal region 116, and sometimes further. For example, the stimulation assembly 118 can extend towards an apical end of the cochlea 140, referred to as the cochlea apex 134. In certain circumstances, the stimulation assembly 118 can be inserted into the cochlea 140 via a cochleostomy 122. In other circumstances, a cochleostomy can be formed through the round window 121, the oval window 112, the promontory 123, or through an apical turn 147 of the cochlea 140.

The elongate stimulation assembly 118 comprises a longitudinally aligned and distally extending array 146 (e.g., electrode array; contact array) of stimulation elements 148 (e.g., electrical electrodes; electrical contacts; optical emitters; optical contacts). The stimulation elements 148 are longitudinally spaced from one another along a length of the elongate body of the stimulation assembly 118. For example, the stimulation assembly 118 can comprise an array 146 comprising twenty-two (22) stimulation elements 148 that are configured to deliver stimulation to the cochlea 140. Although the array 146 of stimulation elements 148 can be disposed on the stimulation assembly 118, in most practical applications, the array 146 is integrated into the stimulation assembly 118 (e.g., the stimulation elements 148 of the array 146 are disposed in the stimulation assembly 118). As noted, the stimulator unit 120 generates stimulation signals (e.g., electrical signals; optical signals) which are applied by the stimulation elements 148 to the cochlea 140, thereby stimulating the auditory nerve 114.

While FIG. 1 schematically illustrates an auditory prosthesis 100 utilizing an external component 142 comprising an external microphone 124, an external sound processing unit 126, and an external power source, in certain other implementations, one or more of the microphone 124, sound processing unit 126, and power source are implantable on or within the recipient (e.g., within the internal component 144). For example, the auditory prosthesis 100 can have each of the microphone 124, sound processing unit 126, and power source implantable on or within the recipient (e.g., encapsulated within a biocompatible assembly located subcutaneously), and can be referred to as a totally implantable cochlear implant (“TICI”). For another example, the auditory prosthesis 100 can have most components of the cochlear implant (e.g., excluding the microphone, which can be an in-the-ear-canal microphone) implantable on or within the recipient, and can be referred to as a mostly implantable cochlear implant (“MICI”).

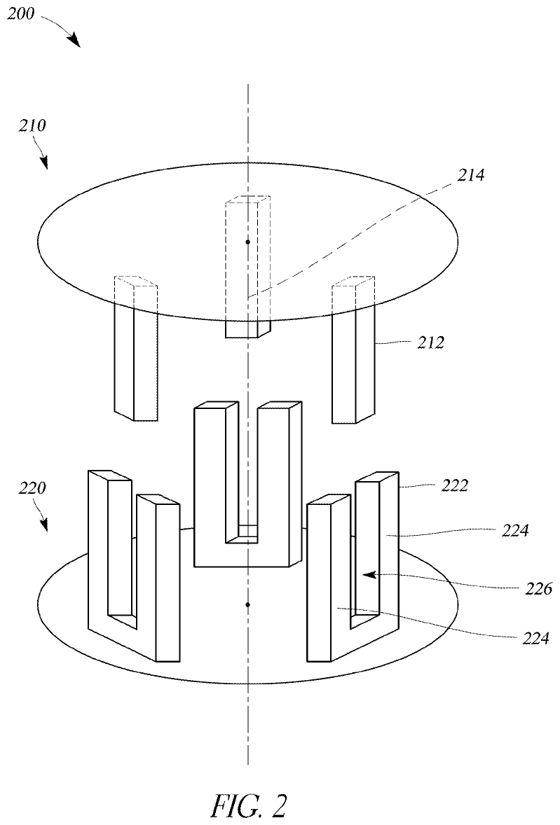

FIG. 2 schematically illustrates an example apparatus 200 in accordance with certain implementations described herein. The apparatus 200 comprises a first portion 210 comprising at least three protrusions 212 in electrical communication with first circuitry (not shown). Each of the at least three protrusions 212 is displaced from and extending substantially parallel to a center axis 214. The apparatus 200 further comprises a second portion 220 configured to be repeatedly mechanically coupled to and decoupled from the first portion 210. The second portion 220 comprises at least three receptacles 222 in electrical communication with second circuitry (not shown). Each of the at least three receptacles 222 comprises two tines 224 configured to be in mechanical and electrical communication with a corresponding protrusion 212 of the at least three protrusions 212 upon insertion of the corresponding protrusion 212 into a region 226 at least partially bounded by the two tines 224. The two tines 224 are displaced from and extending substantially parallel to the center axis 214 and are configured to inhibit a relative rotation between the first portion 210 and the second portion 220 about the center axis 214.

In certain implementations, the at least three protrusions 212 comprise at least one first electrically conductive material (e.g., gold-plated hardened beryllium-copper alloy, for example, BeCu alloy that has previously undergone an age hardening heat treatment subsequently coated with Au) and the two tines 224 of each of the at least three receptacles 222 comprise at least one second electrically conductive material (e.g., gold-plated beryllium-copper alloy) that can be the same as or different from the at least one first electrically conductive material. In certain implementations, each protrusion 212 of the at least three protrusions 212 has a width less than 1 millimeter in a plane substantially perpendicular to the center axis 214 and is displaced from the center axis 214 by a distance less than 2 millimeters. In certain implementations, the at least three protrusions 212 are separated from one another by a center-to-center distance less than or equal to 2 millimeters. In certain implementations, each receptacle 222 of the at least three receptacles 222 has an edge-to-edge width less than 2 millimeters in the plane substantially perpendicular to the center axis 214. The region 226 between the two tines 224 of the receptacle 22 can have an edge-to-edge width less than 1 millimeter and substantially equal to the width of the protrusion 212 configured to be received by the region 226. Various numbers of protrusions 212 (e.g., 3, 4, 5, 6, or more) of the first portion 210 and numbers of corresponding receptacles 222 (e.g., 3, 4, 5, 6, or more) of the second portion 220 are compatible with certain implementations described herein. While various configurations are described herein with each protrusion 212 of the plurality of protrusions 212 being a component of the first portion 210 and each receptacle 222 of the plurality of receptacles 222 being a component of the second portion 220, other configurations in which at least some of the protrusions 212 are components of the second portion 220 and at least some of the receptacles 222 are components of the first portion 210 are also compatible with certain implementations described herein.

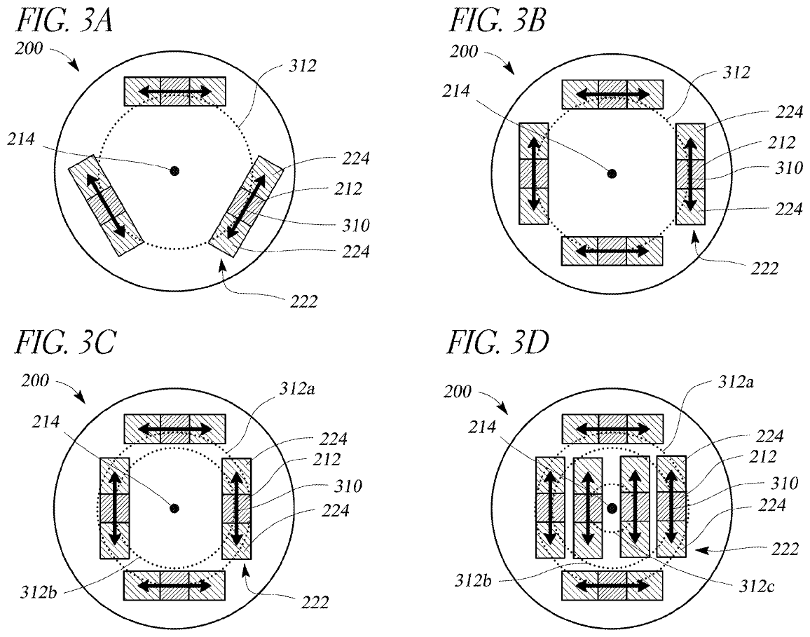

FIGS. 3A-3D schematically illustrate top views of example apparatus 200 along the center axis 214 in accordance with certain implementations described herein. In certain implementations, the two tines 224 of a receptacle 222 are positioned along a line 310 substantially tangent to a circle 312 encircling (e.g., centered on) the center axis 214 and having a radius substantially equal to a corresponding distance of the corresponding protrusion 212 from the center axis 214. For example, as schematically illustrated in FIG. 3A, three protrusions 212 are equidistant from the center axis 214 and the two tines 224 of each of three receptacles 222 are positioned along a corresponding line 310 substantially tangent to the circle 312. For another example, as schematically illustrated in FIG. 3B, four protrusions 212 are equidistant from the center axis 214 and the two tines 224 of each of four receptacles 222 are positioned along a corresponding line 310 substantially tangent to the circle 312.

In certain implementations, at least two of the at least three protrusions 212 are displaced from the center axis 214 by distances that are substantially equal to one another. For example, as schematically illustrated by FIGS. 3A and 3B, each of the protrusions 212 can be displaced from the center axis 214 by the same distance. In certain implementations, at least two of the at least three protrusions 212 are displaced from the center axis 214 by distances that are substantially different from one another. In certain implementations, a first sub-set of two or more protrusions 212 can be displaced from the center axis 214 by first distances that are substantially equal to one another, a second sub-set of two or more protrusions 212 can be displaced from the center axis 214 by second distances that are substantially equal to one another, and the second distances can be substantially different from the first distances. In certain implementations, the two tines 224 of each of the at least three receptacles 222 are spring-loaded and configured to clasp the corresponding protrusion 212.

For example, as schematically illustrated by FIG. 3C, two protrusions 212 are equidistantly displaced from the center axis 214 by a first distance and the two tines 224 of the corresponding receptacles 222 are positioned along a corresponding line 310 substantially tangent to a first circle 312a having a first radius substantially equal to the first distance, and two other protrusions 212 are equidistantly displaced from the center axis 214 by a second distance and the two tines 224 of the corresponding receptacles 222 are positioned along a corresponding line 310 substantially tangent to a second circle 312b having a second radius substantially equal to the second distance. For another example, as schematically illustrated by FIG. 3D, two outermost protrusions 212 are equidistantly displaced from the center axis 214 by a first distance that is substantially equal to a first radius of a first circle 312a, two other protrusions 212 are equidistantly displaced from the center axis 214 by a second distance substantially equal to a second radius of a second circle 312b, and two innermost protrusions 212 are equidistantly displaced from the center axis 214 by a third distance substantially equal to a third radius of a third circle 312c. The tines 224 of the receptacles 222 corresponding to the two outermost protrusions 212 are positioned along a corresponding line 310 substantially tangent to the first circle 312a, the tines 224 of the receptacles 222 corresponding to the two other protrusions 212 are positioned along a corresponding line 310 substantially tangent to the second circle 312b, and the tines 224 of the receptacles 222 corresponding to the two innermost protrusions 212 are positioned along a corresponding line 310 substantially tangent to the third circle 312c. In still another example, each of the protrusions 212 can be displaced from the center axis 214 by corresponding distances that are substantially different from one another. In certain implementations (see, e.g., FIG. 3D), the two tines 224 of each receptacle 222 are configured to not completely encircle the corresponding protrusion 212 upon being coupled to the protrusion 212 (e.g., only a portion of the perimeter of the protrusion 212 is contacted by the tines 224), thereby allowing the receptacles 222 to be positioned closer together such that more receptacles 222 can be fit within the second portion 220 without creating electrical shorts between the receptacles 222 (e.g., allowing the apparatus 200 to have a smaller width perpendicular to the center axis 214 than the width of an apparatus with the receptacles fully encircling the corresponding protrusions).

In certain implementations, the protrusions 212 and the receptacles 222 are configured to resist relative torques between the first portion 210 and the second portion 220 about the center axis 214. For example, such relative torques can be externally applied when the first portion 210 and/or the second portion 220 is twisted as the first and second portions 210, 220 are being coupled together. As schematically illustrated by FIGS. 3A-3D, upon an externally-applied relative torque between the first portion 210 and the second portion 220 about the center axis 214, each protrusion 212 applies a force (denoted in FIGS. 3A-3D by the double-headed arrows of the lines 310) against one of the two tines 224 in contact with the protrusion 212, and the tine 224 applies a counterforce against the protrusion 212. In certain such implementations, the rigidities of the protrusions 212 and the tines 222 inhibit (e.g., prevent; block) the movement of the protrusions 212 relative to the tines 222 along the corresponding circles 312, thereby providing torques that counteract the externally-applied relative torque between the first portion 210 and the second portion 220.

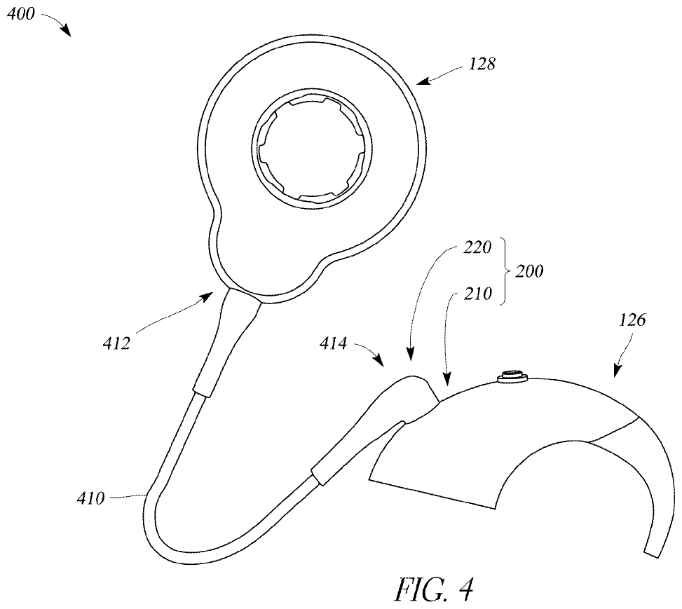

In certain implementations, the apparatus 200 is an external portion of a medical system (e.g., a portion that is not implanted on or within the recipient). For example, FIG. 4 schematically illustrates an example external portion 400 of an acoustic prosthesis system 100 (e.g., a cochlear implant system) comprising an example apparatus 200 in accordance with certain implementations described herein. The portion 400 can comprise a sound processing unit 126 comprising first circuitry (e.g., a power source and/or a processing module) configured to perform signal processing operations. The portion 400 can further comprise an external transmitter unit 128 comprising second circuitry (e.g., at least one external inductive communication coil 130) that is part of an inductive RF communication link with an internal component 144 of the acoustic prosthesis system 100. The external portion 400 of FIG. 4 further comprises an electrical cable 410 (e.g., comprising a plurality of electrically conductive wires) configured to be in electrical communication with the external transmitter unit 128 (e.g., via an electrical connector 412) and in electrical communication with the sound processing unit 126 (e.g., via an electrical connector 414). In certain implementations, one or both of the electrical connectors 412, 414 comprises an apparatus 200 as described herein. In certain implementations (as shown in FIG. 4), a component comprising active circuitry (e.g., the sound processing unit 126) comprises the first portion 210 of the apparatus 200 and the electrical cable 410 comprises the second portion 220 of the apparatus 200, while in certain other implementations, the electrical cable 410 comprises the first portion 210 of the apparatus 200 and the component comprising active circuitry comprises the second portion 220 of the apparatus 200.

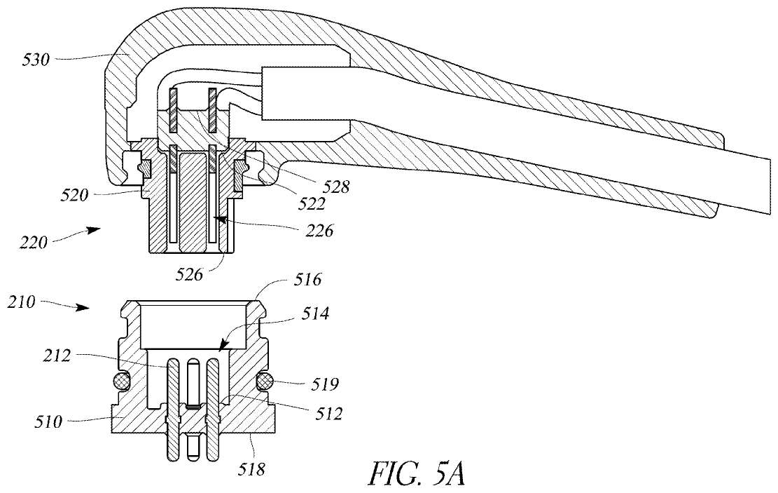

FIGS. 5A-5B schematically illustrate substantially cross-sectional views of an example apparatus 200 with the first portion 210 and the second portion 220 mechanically decoupled from one another and mechanically coupled to one another, respectively, in accordance with certain implementations described herein. In certain implementations, the first portion 210 comprises a socket 510 and the second portion 220 comprises a plug 520 (see, e.g., FIGS. 5A-5B), while in certain other implementations, the first portion 210 comprises a plug 520 and the second portion 220 comprises a socket 510.

As schematically illustrated by FIG. 5A, the protrusions 212 (e.g., pins) of certain implementations comprise first portions that extend from an inner surface 512 of the socket 510 into a region 514 configured to receive the plug 520. In certain implementations (see, e.g., FIG. 5A), the protrusions 212 do not extend past a first outer surface 516 of the socket 510, while in certain other implementations, the protrusions 212 do extend past the first outer surface 516. In certain implementations (see, e.g., FIG. 5A), one or more of the protrusions 212 comprises a second portion that extends from a second outer surface 518 of the socket 510 and that is in electrical communication with electrical conductors (e.g., bonded, soldered, or welded to wires) in electrical communication with the first circuitry.

In certain implementations, the socket 510 is configured to be mounted on or within a component (e.g., the sound processing unit 126) comprising the first circuitry that is in electrical communication with the protrusions 212 (e.g., the first circuitry connected to the second portions of the protrusions 212 extending from the second outer surface 518 of the socket 510). In certain such implementations, the socket 510 is mounted with a moisture-resistant seal between the socket 510 and the surrounding component (e.g., a seal formed by compression of an O-ring 519 between surfaces of the socket 510 and the component).

As schematically illustrated by FIG. 5A, the receptacles 222 (e.g., forks) of certain implementations comprise first portions that comprise the tines 224 (e.g., prongs) and that extend from an inner surface 522 of the plug 520, with the tines 224 at least partially bounding the region 226 within the plug 520 configured to receive a corresponding protrusion 212. In certain implementations (see, e.g., FIG. 5A), the receptacles 222 do not extend past a first outer surface 526 of the plug 520, while in certain other implementations, the receptacles 222 do extend past the first outer surface 526. As schematically illustrated in FIG. 5B, the first outer surface 526 of the plug 520 can be configured to contact or be close to the first outer surface 512 of the socket 510 upon the first and second portions 210, 220 being coupled to one another. In certain implementations (see, e.g., FIG. 5A), one or more of the receptacles 222 comprises a second portion that extends from a second outer surface 528 of the plug 520 (e.g., within a housing 530) to be in electrical communication with electrical conductors (e.g., bonded, soldered, welded to wires) in electrical communication with the second circuitry.

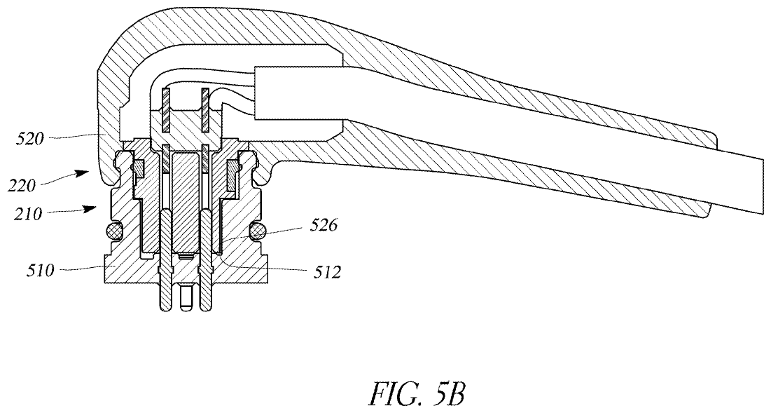

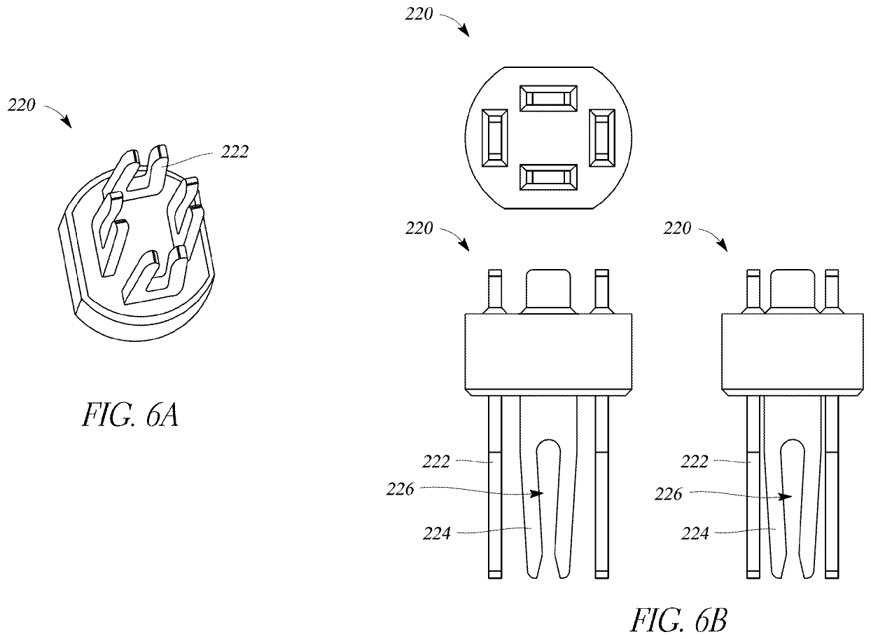

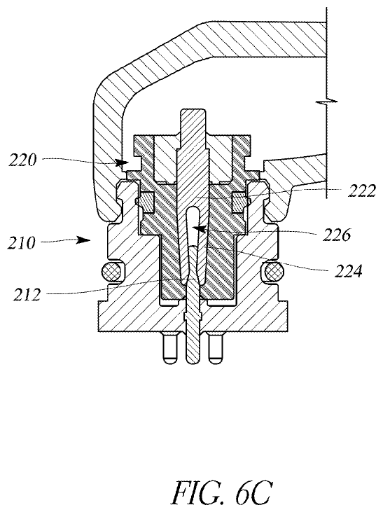

FIG. 6A schematically illustrates a perspective view of an example second portion 220 comprising four receptacles 222 in accordance with certain implementations described herein. FIG. 6B schematically illustrates three side views of the example second portion 220 of FIG. 6A. FIG. 6C schematically illustrates a substantially cross-sectional view of the example second portion 220 of FIGS. 6A-6B in a plane extending along an example protrusion 212 and an example receptacle 222 in mechanical and electrical communication with one another in accordance with certain implementations described herein. The example second portion 220 of FIGS. 6A-6C corresponds to the example apparatus 200 of FIG. 3C in which two of the four receptacles 222 are each a first distance from the center axis 214 and the other two of the four receptacles 222 are each a second distance from the center axis 214, the second distance substantially different from the first distance.

As schematically illustrated by FIGS. 6A-6B, the second portion 220 can comprise a plurality of electrically conductive receptacles 222 (e.g., forks) spaced from and distributed about an axis (e.g., center axis 214). Each receptacle 222 of the plurality of receptacles 222 has a pair of substantially parallel tines 224 (e.g., prongs) configured to be repeatedly mechanically and electrically engaged with and disengaged from a corresponding electrically conductive pin (e.g., protrusion 212 of the first portion 210). The tines 224 extend substantially parallel to the axis and are spaced from one another along a line substantially perpendicular to the axis and substantially perpendicular to a direction extending from the axis to the receptable 222. In certain implementations, each receptacle 222 of the plurality of receptacles 222 is substantially identical to one another.

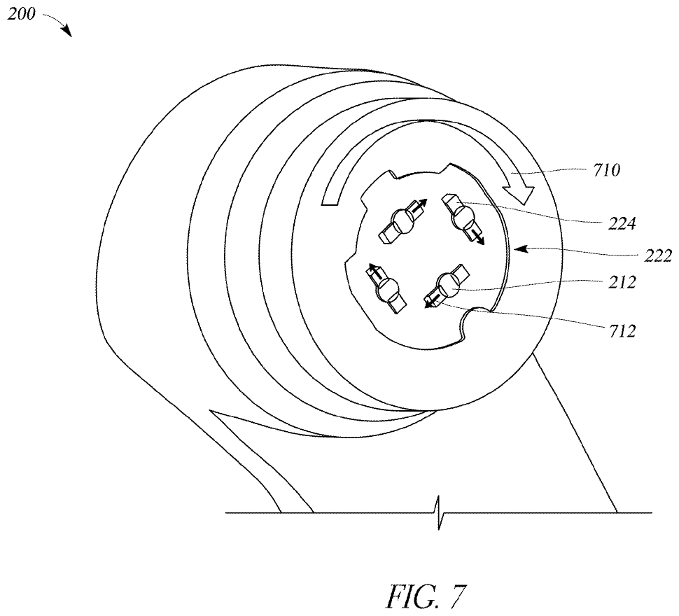

FIG. 7 schematically illustrates a substantially cross-sectional view in a plane substantially perpendicular to a center axis 214 extending through an example apparatus 200 in accordance with certain implementations described herein. An externally-applied relative torque between the first and second portions 210, 220 about the center axis 214 is denoted by arrow 710, and a resulting force applied by the protrusions 212 onto the tines 224 of the receptacles 222 are denoted by arrows 712. In certain such implementations, the tines 222 are configured to inhibit (e.g., prevent; block) the movement of the protrusions 212 relative to the tines 222 due to the externally-applied torque, and the resulting counterforces applied by the tines 222 to the protrusions 212 result in torques that counteract the externally-applied relative torque between the first portion 210 and the second portion 220.

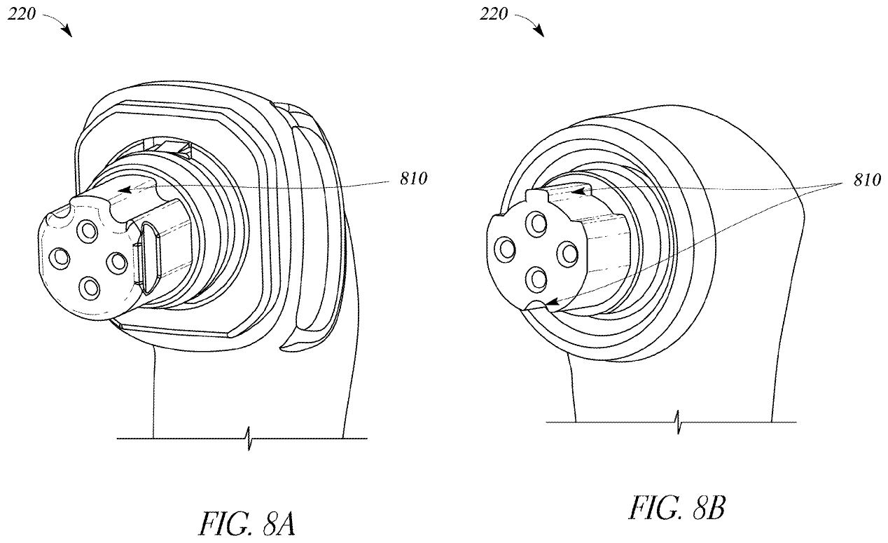

FIGS. 8A and 8B schematically illustrate two example second portions 220 having at least one rotation inhibiting structure 810 in accordance with certain implementations described herein. For example, the first portion 210 can comprise at least one first interlock portion (e.g., at least one socket portion having one or more recesses and/or protrusions) and the second portion 220 comprises at least one second interlock portion (e.g., at least one plug portion having one or more protrusions and/or recesses) configured to couple (e.g., mate; engage) with and to decouple (e.g., disengage) from the at least one first interlock portion. The at least one first interlock portion and the at least one second interlock portion can be configured to inhibit the relative rotation between the first portion 210 and the second portion 220 about the center axis 214 such that the at least one rotation inhibiting structure 810 provides additional protection against externally-applied relative torques between the first portion 210 and the second portion 220.

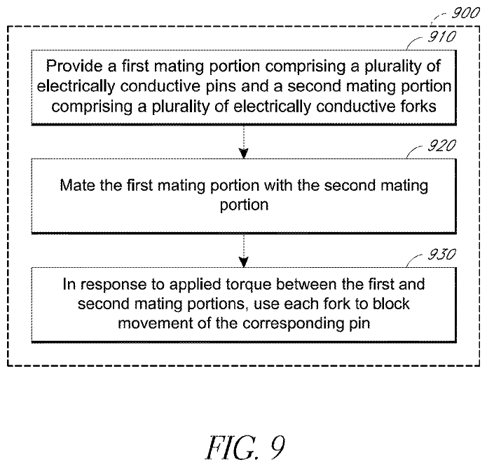

FIG. 9 is a flow diagram of an example method 900 in accordance with certain implementations described herein. In an operational block 910, the method 900 comprises providing a first mating portion (e.g., first portion 210) comprising a plurality of electrically conductive pins (e.g., protrusions 212) and a second mating portion (e.g., second portion 220) comprising a plurality of electrically conductive forks (e.g., receptacles 222) configured to receive the plurality of electrically conductive pins. For example, the first mating portion can comprise a socket 510 of an electrical connector and the second mating portion can comprise a plug 520 of the electrical connector.

In an operational block 920, the method 900 further comprises mating the first mating portion with the second mating portion such that each of the forks of the plurality of forks is in electrical and mechanical communication with a corresponding pin of the plurality of pins. In an operational block 930, the method 900 further comprises, in response to an applied torque between the first and second mating portions, using each fork to block movement of the corresponding pin. For example, each fork can generate a torque that at least partially counters the applied torque.

Although commonly used terms are used to describe the systems and methods of certain implementations for ease of understanding, these terms are used herein to have their broadest reasonable interpretations. Although various aspects of the disclosure are described with regard to illustrative examples and implementations, the disclosed examples and implementations should not be construed as limiting. Conditional language, such as, among others, “can,” “could,” “might,” or “may,” unless specifically stated otherwise, or otherwise understood within the context as used, is generally intended to convey that certain implementations include, while other implementations do not include, certain features, elements and/or steps. Thus, such conditional language is not generally intended to imply that features, elements and/or steps are in any way required for one or more implementations or that one or more implementations necessarily include logic for deciding, with or without user input or prompting, whether these features, elements and/or steps are included or are to be performed in any particular implementation. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

It is to be appreciated that the implementations disclosed herein are not mutually exclusive and may be combined with one another in various arrangements. In addition, although the disclosed methods and apparatuses have largely been described in the context of conventional cochlear implants, various implementations described herein can be incorporated in a variety of other suitable devices, methods, and contexts. More generally, as can be appreciated, certain implementations described herein can be used in a variety of wearable device contexts that can utilize small electrical connectors comprising multiple portions that are configured to be repeatedly coupled to and decoupled from one another.

Language of degree, as used herein, such as the terms “approximately,” “about,” “generally,” and “substantially,” represent a value, amount, or characteristic close to the stated value, amount, or characteristic that still performs a desired function or achieves a desired result. For example, the terms “approximately,” “about,” “generally,” and “substantially” may refer to an amount that is within ±10% of, within ±5% of, within ±2% of, within ±1% of, or within ±0.1% of the stated amount. As another example, the terms “generally parallel” and “substantially parallel” refer to a value, amount, or characteristic that departs from exactly parallel by ±10 degrees, by ±5 degrees, by ±2 degrees, by ±1 degree, or by ±0.1 degree, and the terms “generally perpendicular” and “substantially perpendicular” refer to a value, amount, or characteristic that departs from exactly perpendicular by ±10 degrees, by ±5 degrees, by ±2 degrees, by ±1 degree, or by ±0.1 degree. The ranges disclosed herein also encompass any and all overlap, sub-ranges, and combinations thereof. Language such as “up to,” “at least,” “greater than,” less than,” “between,” and the like includes the number recited. As used herein, the meaning of “a,” “an,” and “said” includes plural reference unless the context clearly dictates otherwise. Also, as used in the description herein, the meaning of “in” includes “into” and “on,” unless the context clearly dictates otherwise.

While the methods and systems are discussed herein in terms of elements labeled by ordinal adjectives (e.g., first, second, etc.), the ordinal adjective are used merely as labels to distinguish one element from another (e.g., one signal from another or one circuit from one another), and the ordinal adjective is not used to denote an order of these elements or of their use.

The invention described and claimed herein is not to be limited in scope by the specific example implementations herein disclosed, since these implementations are intended as illustrations, and not limitations, of several aspects of the invention. Any equivalent implementations are intended to be within the scope of this invention. Indeed, various modifications of the invention in form and detail, in addition to those shown and described herein, will become apparent to those skilled in the art from the foregoing description. Such modifications are also intended to fall within the scope of the claims. The breadth and scope of the invention should not be limited by any of the example implementations disclosed herein, but should be defined only in accordance with the claims and their equivalents.

1. An apparatus comprising:
a first portion comprising at least three protrusions in electrical communication with first circuitry, each of the at least three protrusions displaced from and extending substantially parallel to a center axis, at least two of the at least three protrusions displaced from the center axis by distances that are substantially different from one another; and
a second portion configured to be repeatedly mechanically coupled to and decoupled from the first portion, the second portion comprising at least three receptacles in electrical communication with second circuitry, each of the at least three receptacles comprising two tines configured to be in mechanical and electrical communication with a corresponding protrusion of the at least three protrusions upon insertion of the corresponding protrusion into a region at least partially bounded by the two tines, the two tines displaced from and extending substantially parallel to the center axis and configured to inhibit a relative rotation between the first portion and the second portion about the center axis.

2. The apparatus of claim 1, wherein the first portion comprises a socket and the second portion comprises a plug.

3. The apparatus of claim 1, wherein each protrusion of the at least three protrusions has a width less than 1 millimeter in a plane substantially perpendicular to the center axis and is displaced from the center axis by a distance less than 2 millimeters, the at least three protrusions are separated from one another by a center-to-center distance less than or equal to 2 millimeters.

4. The apparatus of claim 3, wherein each receptacle of the at least three receptacles has a width less than 2 millimeters in the plane substantially perpendicular to the center axis.

5. The apparatus of claim 1, wherein the two tines are spring-loaded and configured to clasp the corresponding protrusion.

6. The apparatus of claim 1, wherein the two tines are positioned along a line substantially tangent to a circle centered on the center axis and having a radius substantially equal to a corresponding distance of the corresponding protrusion from the center axis.

7. The apparatus of claim 1, wherein at least two of the at least three protrusions are displaced from the center axis by distances that are substantially equal to one another.

8. The apparatus of claim 1, wherein the first portion comprises at least one first interlock portion and the second portion comprises at least one second interlock portion configured to engage with and disengage from the at least one first interlock portion, the at least one first interlock portion and the at least one second interlock portion configured to inhibit the relative rotation between the first portion and the second portion about the center axis.

9. The apparatus of claim 1, wherein the apparatus is an external portion of an acoustic prosthesis system comprising:
a sound processing unit comprising the first circuitry; and
an electrical cable configured to be in electrical communication with the second circuitry.

10. An apparatus comprising:
at least three electrically conductive forks spaced from and distributed about an axis, each fork of the at least three forks having a pair of substantially parallel prongs, the prongs of the pair of prongs extending substantially parallel to the axis and spaced from one another along a line substantially perpendicular to the axis and substantially perpendicular to a direction extending from the axis to the fork, at least two of the at least three electrically conductive forks displaced from the axis by distances that are substantially different from one another.

11. The apparatus of claim 10, wherein each fork of the at least three forks is substantially identical to one another.

12. The apparatus of any of claim 10, wherein the prongs comprise hardened BeCu alloy coated with Au.

13. The apparatus of claim 10, wherein each fork of the at least three forks has a width along the line less than 2 millimeters, and the prongs are spaced from one another along the line by a center-to-center distance less than or equal to 1 millimeter.

14. The apparatus of claim 10, further comprising at least three electrically conductive pins spaced from and distributed about the axis and extending substantially parallel to the axis, the at least three pins configured to repeatedly mechanically and electrically engage with and disengage from the at least three forks.

15. The apparatus of claim 14, wherein each pin of the at least three pins has a width less than 1 millimeter in a plane substantially perpendicular to the axis and is displaced from the axis by a distance less than 2 millimeters, the at least three pins separated from one another by a center-to-center distance less than or equal to 2 millimeters.

16. The apparatus of claim 14, wherein, upon an externally-applied relative torque between the at least three forks and the at least three pins about the axis, the at least three pins apply a force against the at least three forks, and the at least three forks apply a counterforce against the at least three pins.

17. A method comprising:
providing a first mating portion comprising a plurality of electrically conductive pins and a second mating portion comprising a plurality of electrically conductive forks configured to receive the plurality of electrically conductive pins;
mating the first mating portion with the second mating portion such that each of the forks of the plurality of forks is in electrical and mechanical communication with a corresponding pin of the plurality of pins; and
in response to an applied torque between the first and second mating portions, using each fork to block movement of the corresponding pin.

18. The method of claim 17, wherein the first mating portion comprises a socket of an electrical connector and the second mating portion comprises a plug of the electrical connector.

19. The method of claim 17, further comprising using each fork to generate a torque that at least partially counters the applied torque.

20. The method of claim 17, wherein the plurality of electrically conductive pins comprises at least three pins spaced from and extending substantially parallel to an axis, at least two of the at least three pins spaced from the axis by distances that are substantially different from one another.

21. The method of claim 20, wherein the plurality of electrically conductive forks comprises at least three forks spaced from the axis, each fork of the at least three forks having a pair of substantially parallel prongs extending substantially parallel to the axis and spaced from one another along a line substantially perpendicular to the axis and substantially perpendicular to a direction extending from the axis to the fork, at least two of the at least three forks spaced from the axis by distances that are substantially different from one another.
